(19) United States
(12) Patent Application Publication
(10) Pub. No.: US 2014/0100136 A1
(43) Pub. Date: Apr. 10, 2014

(54) ISOLATION AND CHARACTERIZATION OF PATHOGENS

(71) Applicant: NanoMR, Inc., Albuquerque, NM (US)

(72) Inventors: Lisa-Jo A. Clarizia, Albuquerque, NM (US); Eddie W. Adams, Albuquerque, NM (US); Sergey A. Dryga, Albuquerque, NM (US)

(73) Assignee: NanoMR, Inc., Albuquerque, NM (US)

(21) Appl. No.: 14/102,861

(22) Filed: Dec. 11, 2013

Related U.S. Application Data
(63) Continuation-in-part of application No. 12/850,203, filed on Aug. 4, 2010.
(60) Provisional application No. 61/739,647, filed on Dec. 19, 2012.
(60) Provisional application No. 61/326,588, filed on Apr. 21, 2010.

(51) Int. Cl. G01N 33/569; C12Q 1/70; C12Q 1/68

(52) U.S. Cl. CPC G01N 33/56911; C12Q 1/6895; C12Q 1/70; G01N 33/56961; G01N 33/56983; C12Q 1/689
USPC 506/9; 435/6.12; 435/5; 435/6.15; 435/7.1; 435/7.92; 435/6.11

(57) ABSTRACT

Methods of the invention generally involve using magnetic particles to isolate low levels of pathogens from a samples and identifying genes expressed by those pathogen. In one aspect, the method includes obtaining a sample comprising a pathogen, forming magnetic particle/target complexes, separating the magnetic particle/target complexes using magnetic fields, and determining an expression profile of a nucleic acid derived from the target.

### RELATED APPLICATIONS

The present application is a continuation-in-part of U.S. patent application Ser. No. 12/850,203 filed Aug. 4, 2010, which claims the benefit of and priority to U.S. provisional application Ser. No. 61/326,588, filed Apr. 21, 2010. This application also claims the benefit of and priority to U.S. provisional application Ser. No. 61/739,647, filed Dec. 19, 2012. The content of each of the above referenced patent applications is incorporated by reference herein in its entirety.

### TECHNICAL FIELD

The invention generally relates to using magnetic particles to isolate low levels of pathogens from a sample for identification of genes expressed by the pathogen.

### BACKGROUND

Blood-borne pathogens are a significant healthcare problem. A delayed or improper diagnosis of a bacterial infection can result in sepsis, a serious, and often deadly, inflammatory response to the infection. Sepsis is the 10^{th} leading cause of death in the United States. Early detection of bacterial infections in blood is the key to preventing the onset of sepsis. Traditional methods of detection and identification of blood-borne infection include blood culture and antibiotic susceptibility assays. Those methods typically require culturing cells, which can be expensive and can take as long as 72 hours. Often, septic shock will occur before cell culture results can be obtained.

Alternative methods for detection of pathogens, particularly bacteria, have been described by others. Those methods include molecular detection methods, antigen detection methods, and metabolite detection methods. Molecular detection methods, whether involving hybrid capture or polymerase chain reaction (PCR), require high concentrations of purified DNA for detection. Both antigen detection and metabolite detection methods also require a relatively large amount of bacteria and have high limit of detection (usually >10⁴ CFU/mL), thus requiring an enrichment step prior to detection. This incubation/enrichment period is intended to allow for the growth of bacteria and an increase in bacterial cell numbers to more readily aid in identification. In many cases, a series of two or three separate incubations is needed to isolate the target bacteria. However, such enrichment steps require a significant amount of time (e.g., at least a few days to a week) and can potentially compromise test sensitivity by killing some of the cells sought to be measured.

There is a need for methods for isolating targets, such as pathogens and other infections agents, from a sample, such as a blood sample, without an additional enrichment step. There is also a need for methods of isolating target analytes that are fast and sensitive in order to provide data for patient treatment decisions in a clinically relevant time frame.

### SUMMARY

The present invention provides methods for isolating infectious agents, such as pathogens, in a biological sample. The invention allows the rapid detection of pathogens at very low levels in the sample; thus enabling early and accurate detection and identification of the pathogens. In certain aspects, pathogens or other target cells are isolated from large volumes of sample (e.g. whole blood) at levels from about 10 CFU/mL to about 1 CFU/mL. In addition, methods of the invention reduce or eliminate the incubation step that is typically associated with pathogen analysis and allow for more rapid analysis of the isolated pathogen. Thus, methods of the invention greatly expand the ability to analyze and characterize pathogens early on, such as during active blood borne infection, in comparison to conventional pathogen isolation/detection techniques that require a few days to a week of time. Particularly advantageous, methods of the invention provide for identification of the genes expressed by a pathogen at an earlier stage of infection than possible with other techniques.

Identification of genes expressed by a pathogen during active blood-borne infection has several benefits and uses. Determining which genes are expressed by a pathogen helps to identify novel targets for antimicrobial therapy and may elucidate the pathophysiology of infection by the target pathogen. In addition, the gene expression profiles of pathogens allow for identification of transcripts encoding for surface antigens, which can be used for antibody generation. The antibodies can be used for diagnostic purposes, e.g. identification of pathogens, or for immunological purposes, e.g. rational vaccine design.

In certain aspects, a cDNA library of the pathogen isolated during active-blood borne infection is constructed. The cDNA library can be used in antibody screening efforts to identify those proteins most strongly identified by a particular antiserum. Knowing which proteins contribute to antibody responses allows one to generate tailored ‘rational’ vaccines against a smaller subset of antigens. For example, restricting a vaccine to a protein antigen will favor a T-cell response over a humoral B-cell response.

Methods of the invention involve introducing magnetic particles to a biological sample (e.g., a tissue or body fluid sample). The sample is incubated to allow the particles to bind to pathogen in the sample, and a magnetic field is applied to capture pathogen/magnetic particle complexes on a surface. Optionally, the surface can be washed with a wash solution that reduces particle aggregation, thereby isolating pathogen/magnetic particle complexes. A particular advantage of compositions of the invention is for capture and isolation of bacteria and fungi directly from blood samples at low concentrations that are present in many clinical samples (as low as 1 CFU/mL of bacteria in a blood sample). Preferably, the magnetic particles comprise a pathogen binding element that has one or more magnetic particles attached to it.

In certain aspects, methods of the invention involve obtaining a heterogeneous sample including a pathogen, exposing the sample to a cocktail including a plurality of sets of magnetic particles, members of each set being conjugated to an antibody specific for a pathogen, and separating particle bound pathogen from other components in the sample. Methods of the invention may further involve characterizing the pathogen. Characterizing may include identifying the pathogen by any technique known in the art. Exemplary techniques include sequencing nucleic acid derived from the pathogen or amplifying the nucleic acid.

The antibodies conjugated to the particles may be either monoclonal or polyclonal antibodies. Methods of the invention may be used to isolate pathogen from heterogeneous sample. In particular embodiments, the heterogeneous sample is a blood sample.

Since each set of particles is conjugated with antibodies have different specificities for different pathogens, compositions of the invention may be provided such that each set of antibody conjugated particles is present at a concentration designed for detection of a specific pathogen in the sample. In certain embodiments, all of the sets are provided at the same concentration. Alternatively, the sets are provided at different concentrations.

To facilitate detection of the different sets of pathogen/magnetic particle complexes the particles may be differently labeled. Any detectable label may be used with compositions of the invention, such as fluorescent labels, radiolabels, enzymatic labels, and others. In particular embodiments, the detectable label is an optically-detectable label, such as a fluorescent label. Exemplary fluorescent labels include Cy3, Cy5, Atto, cyanine, rhodamine, fluorescien, coumarin, BODIPY, alexa, and conjugated multi-dyes.

Methods of the invention may be used to isolate only gram positive bacteria from a sample. Alternatively, methods of the invention may be used to isolate only gram negative bacteria from a sample. In certain embodiments, methods of the invention are used to isolate both gram positive and gram negative bacteria from a sample. In still other embodiments, methods isolate specific pathogen from a sample. Exemplary bacterial species that may be captured and isolated by methods of the invention include E. coli, Listeria, Clostridium, Mycobacterium, Shigella, Borrelia, Campylobacter, Bacillus, Salmonella, Staphylococcus, Enterococcus, Pneumococcus, Streptococcus, and a combination thereof.

Methods of the invention are not limited to isolating pathogens from a body fluid. Methods of the invention may be designed to isolate other types of target analytes, such as fungi, protein, a cell, a virus, a nucleic acid, a receptor, a ligand, or any molecule known in the art.

Compositions used in methods of the invention may use any type of magnetic particle. Magnetic particles generally fall into two broad categories. The first category includes particles that are permanently magnetizable, or ferromagnetic; and the second category includes particles that demonstrate bulk magnetic behavior only when subjected to a magnetic field. The latter are referred to as magnetically responsive particles. Materials displaying magnetically responsive behavior are sometimes described as superparamagnetic. However, materials exhibiting bulk ferromagnetic properties, e.g., magnetic iron oxide, may be characterized as superparamagnetic when provided in crystals of about 30 nm or less in diameter. Larger crystals of ferromagnetic materials, by contrast, retain permanent magnet characteristics after exposure to a magnetic field and tend to aggregate thereafter due to strong particle-particle interaction. In certain embodiments, the particles are superparamagnetic particles. In other embodiments, the magnetic particles include at least 70% superparamagnetic particles by weight. In certain embodiments, the superparamagnetic particles are from about 100 nm to about 250 nm in diameter. In certain embodiments, the magnetic particle is an iron-containing magnetic particle. In other embodiments, the magnetic particle includes iron oxide or iron platinum.

Another aspect of the invention provides methods for isolating pathogen from a heterogeneous sample, that involve labeling pathogen from a biological sample with a cocktail including a plurality of sets of magnetic particles, members of each set being conjugated to an antibody specific for a pathogen, exposing the sample to a magnetic field to isolate pathogen conjugated to the particles, and isolating particle bound pathogen from other components of the sample. Methods of the invention may further involve eluting pathogens from the particles.

Methods of the invention may further involve characterizing the pathogen. Characterizing may include identifying the pathogen by any technique known in the art. Exemplary techniques include conducting an assay to determine the expression profile of nucleic acid derived from the pathogen or amplifying the nucleic acid. For example, after capture, the pathogen is lysed and messenger RNA transcripts are converted into cDNA. The cDNA is then hybridized to DNA microarrays to obtain both the identity of the transcript and the relative expression level. Such microarray analysis is useful for studying the physiology of the pathogen as exposed to, for example, human blood and the human immune system. The generated cDNA can also be sub-cloned into a bacterial expression vector to construct a cDNA library.

In another aspect, the assay for isolating target analytes or pathogens involves applying alternating a magnetic field. In such aspect, methods the invention involve contacting a sample with magnetic particles including first moieties specific for a target analyte, thereby forming target/particle complexes in the sample, flowing the sample through a channel including second moieties attached to at least one surface of the channel, applying magnetic field to the flowing sample to result in target/particle complexes being brought into proximity of the surface to bind the second moieties and unbound particles remaining free in the sample, binding the target/particle complexes to the second moieties, and washing away unbound particles and unbound analytes of the sample. The magnetic field assists in ensuring the pathogens bind to the second moieties and are separated from other components of the sample.

### BRIEF DESCRIPTION OF THE DRAWINGS

FIG. 1 provides one exemplary configuration of a flow cell and first and second sets of magnets for generating alternative magnetic fields.

FIG. 2A provides an exemplary process chart for implementation of methods of the invention for separation of bacteria from blood. FIG. 2B provides a magnified view of a target/magnetic particle complex.

### DETAILED DESCRIPTION

The invention generally relates to conducting an assay on a sample that isolates a bacterium from the sample. In certain embodiments, the assay isolates as low as 1 CFU/mL of bacteria in the sample. In certain aspects, methods of the invention isolate as low as 1 CFU/mL of bacteria in the sample by using magnetic particles having a particular magnetic moment (as determined by particle size and % weight of magnetic material) that capture target pathogens in a body fluid sample and magnets to isolate the target. Once isolated, methods of the invention provide for analyzing the captured target analyte to identify which genes are expressed by the pathogen.

In certain aspects, methods of the invention involve introducing magnetic particles including a target-specific binding moiety to a body fluid sample in order to create a mixture, incubating the mixture to allow the particles to bind to a target, applying a magnetic field to capture target/magnetic particle complexes on a surface, thereby isolating target/magnetic particle complexes. Methods of the invention may further involve washing the mixture in a wash solution that reduces particle aggregation. Certain fundamental technologies and principles are associated with binding magnetic materials to target entities and subsequently separating by use of magnet fields and gradients. Such fundamental technologies and principles are known in the art and have been previously described, such as those described in Janeway (Immunobiology, 6^{th} edition, Garland Science Publishing), the content of which is incorporated by reference herein in its entirety.

Methods of the invention involve collecting a sample, such as a tissue or body fluid. The sample may be collected in any clinically acceptable manner. A body fluid having a target can be collected in a container, such as a blood collection tube (e.g., VACUTAINER, test tube specifically designed for venipuncture, commercially available from Becton, Dickinson and company). In certain embodiments, a solution is added that prevents or reduces aggregation of endogenous aggregating factors, such as heparin in the case of blood.

A body fluid refers to a liquid material derived from, for example, a human or other mammal. Such body fluids include, but are not limited to, mucus, blood, plasma, serum, serum derivatives, bile, phlegm, saliva, sweat, amniotic fluid, mammary fluid, urine, sputum, and cerebrospinal fluid (CSF), such as lumbar or ventricular CSF. A body fluid may also be a fine needle aspirate. A body fluid also may be media containing cells or biological material. In particular embodiments, the fluid is blood.

A tissue is a mass of connected cells and/or extracellular matrix material, e.g. skin tissue, nasal passage tissue, CNS tissue, neural tissue, eye tissue, liver tissue, kidney tissue, placental tissue, mammary gland tissue, placental tissue, gastrointestinal tissue, musculoskeletal tissue, genitourinary tissue, bone marrow, and the like, derived from, for example, a human or other mammal and includes the connecting material and the liquid material in association with the cells and/or tissues. A sample may also be a fine needle aspirate or biopsied tissue. A sample also may be media containing cells or biological material.

Methods of the invention may be used to detect any target. The target refers to the substance in the sample that will be captured and isolated by methods of the invention. The target may be bacteria, fungi, a protein, a cell (such as a cancer cell, a white blood cell a virally infected cell, or a fetal cell circulating in maternal circulation), a virus, a nucleic acid (e.g., DNA or RNA), a receptor, a ligand, a hormone, a drug, a chemical substance, or any molecule known in the art. In certain embodiments, the target is a pathogenic bacteria. In other embodiments, the target is a gram positive or gram negative bacteria. Exemplary bacterial species that may be captured and isolated by methods of the invention include E. coli, Listeria, Clostridium, Mycobacterium, Shigella, Borrelia, Campylobacter, Bacillus, Salmonella, Staphylococcus, Enterococcus, Pneumococcus, Streptococcus, and a combination thereof. A particular advantage of methods of the invention is for capture and isolation of bacteria and fungi directly from blood samples at low concentrations that are present in many clinical samples (as low as 1 CFU/mL of bacteria in a blood sample).

The sample is then mixed with magnetic particles having a particular magnetic moment and also including a target-specific binding moiety to generate a mixture that is allowed to incubate such that the particles bind to a target in the sample, such as a bacterium in a blood sample. The mixture is allowed to incubate for a sufficient time to allow for the particles to bind to the target. The process of binding the magnetic particles to the targets associates a magnetic moment with the targets, and thus allows the targets to be manipulated through forces generated by magnetic fields upon the attached magnetic moment.

In general, incubation time will depend on the desired degree of binding between the target and the magnetic particles (e.g., the amount of moment that would be desirably attached to the target), the amount of moment per target, the amount of time of mixing, the type of mixing, the reagents present to promote the binding and the binding chemistry system that is being employed. Incubation time can be anywhere from about 5 seconds to a few days. Exemplary incubation times range from about 10 seconds to about 2 hours. Binding occurs over a wide range of temperatures, generally between 15° C. and 40° C.

Methods of the invention are performed with magnetic particles having a magnetic moment that allows for isolation of as low as 1 CFU/mL of bacteria in the sample. Production of magnetic particles is shown for example in Giaever (U.S. Pat. No. 3,970,518), Senyi et al. (U.S. Pat. No. 4,230,685), Dodin et al. (U.S. Pat. No. 4,677,055), Whitehead et al. (U.S. Pat. No. 4,695,393), Benjamin et al. (U.S. Pat. No. 5,695,946), Giaever (U.S. Pat. No. 4,018,886), Rembaum (U.S. Pat. No. 4,267,234), Molday (U.S. Pat. No. 4,452,773), Whitehead et al. (U.S. Pat. No. 4,554,088), Forrest (U.S. Pat. No. 4,659,678), Liberti et al. (U.S. Pat. No. 5,186,827), Own et al. (U.S. Pat. No. 4,795,698), and Liberti et al. (WO 91/02811), the content of each of which is incorporated by reference herein in its entirety.

Magnetic particles generally fall into two broad categories. The first category includes particles that are permanently magnetizable, or ferromagnetic; and the second category includes particles that demonstrate bulk magnetic behavior only when subjected to a magnetic field. The latter are referred to as magnetically responsive particles. Materials displaying magnetically responsive behavior are sometimes described as superparamagnetic. However, materials exhibiting bulk ferromagnetic properties, e.g., magnetic iron oxide, may be characterized as superparamagnetic when provided in crystals of about 30 nm or less in diameter. Larger crystals of ferromagnetic materials, by contrast, retain permanent magnet characteristics after exposure to a magnetic field and tend to aggregate thereafter due to strong particle-particle interaction. In certain embodiments, the particles are superparamagnetic beads. In certain embodiments, the magnetic particle is an iron containing magnetic particle. In other embodiments, the magnetic particle includes iron oxide or iron platinum.

In certain embodiments, the magnetic particles include at least about 10% superparamagnetic beads by weight, at least about 20% superparamagnetic beads by weight, at least about 30% superparamagnetic beads by weight, at least about 40% superparamagnetic beads by weight, at least about 50% superparamagnetic beads by weight, at least about 60% superparamagnetic beads by weight, at least about 70% superparamagnetic beads by weight, at least about 80% superparamagnetic beads by weight, at least about 90% superparamagnetic beads by weight, at least about 95% superparamagnetic beads by weight, or at least about 99% superparamagnetic beads by weight. In a particular embodiment, the magnetic particles include at least about 70% superparamagnetic beads by weight.

In certain embodiments, the superparamagnetic beads are less than 100 nm in diameter. In other embodiments, the superparamagnetic beads are about 150 nm in diameter, are about 200 nm in diameter, are about 250 nm in diameter, are about 300 nm in diameter, are about 350 nm in diameter, are about 400 nm in diameter, are about 500 nm in diameter, or are about 1000 nm in diameter. In a particular embodiment, the superparamagnetic beads are from about 100 nm to about 250 nm in diameter.

In certain embodiments, the particles are beads (e.g., nanoparticles) that incorporate magnetic materials, or magnetic materials that have been functionalized, or other configurations as are known in the art. In certain embodiments, nanoparticles may be used that include a polymer material that incorporates magnetic material(s), such as nanometal material(s). When those nanometal material(s) or crystal(s), such as Fe₃O₄, are superparamagnetic, they may provide advantageous properties, such as being capable of being magnetized by an external magnetic field, and demagnetized when the external magnetic field has been removed. This may be advantageous for facilitating sample transport into and away from an area where the sample is being processed without undue bead aggregation.

One or more or many different nanometal(s) may be employed, such as Fe₃O₄, FePt, or Fe, in a core-shell configuration to provide stability, and/or various others as may be known in the art. In many applications, it may be advantageous to have a nanometal having as high a saturated moment per volume as possible, as this may maximize gradient related forces, and/or may enhance a signal associated with the presence of the beads. It may also be advantageous to have the volumetric loading in a bead be as high as possible, for the same or similar reason(s). In order to maximize the moment provided by a magnetizable nanometal, a certain saturation field may be provided. For example, for Fe₃O₄ superparamagnetic particles, this field may be on the order of about 0.3 T.

The size of the nanometal containing bead may be optimized for a particular application, for example, maximizing moment loaded upon a target, maximizing the number of beads on a target with an acceptable detectability, maximizing desired force-induced motion, and/or maximizing the difference in attached moment between the labeled target and non-specifically bound targets or bead aggregates or individual beads. While maximizing is referenced by example above, other optimizations or alterations are contemplated, such as minimizing or otherwise desirably affecting conditions.

In an exemplary embodiment, a polymer bead containing 80 wt % Fe₃O₄ superparamagnetic particles, or for example, 90 wt % or higher superparamagnetic particles, is produced by encapsulating superparamagnetic particles with a polymer coating to produce a bead having a diameter of about 250 nm.

Magnetic particles for use with methods of the invention have a target-specific binding moiety that allows for the particles to specifically bind the target of interest in the sample. The target-specific moiety may be any molecule known in the art and will depend on the target to be captured and isolated. Exemplary target-specific binding moieties include nucleic acids, proteins, ligands, antibodies, aptamers, and receptors.

In particular embodiments, the target-specific binding moiety is an antibody, such as an antibody that binds a particular bacterium. General methodologies for antibody production, including criteria to be considered when choosing an animal for the production of antisera, are described in Harlow et al. (Antibodies, Cold Spring Harbor Laboratory, pp. 93-117, 1988). For example, an animal of suitable size such as goats, dogs, sheep, mice, or camels are immunized by administration of an amount of immunogen, such the target bacteria, effective to produce an immune response. An exemplary protocol is as follows: the animal is injected with 100 milligrams of antigen resuspended in adjuvant, for example Freund's complete adjuvant, dependent on the size of the animal, followed three weeks later with a subcutaneous injection of 100 micrograms to 100 milligrams of immunogen with adjuvant dependent on the size of the animal, for example Freund's incomplete adjuvant. Additional subcutaneous or intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the dilution having a detectable activity. The antibodies are purified, for example, by affinity purification on columns containing protein G resin or target-specific affinity resin.

The technique of in vitro immunization of human lymphocytes is used to generate monoclonal antibodies. Techniques for in vitro immunization of human lymphocytes are well known to those skilled in the art. See, e.g., Inai, et al., Histochemistry, 99(5):335 362, May 1993; Mulder, et al., Hum. Immunol., 36(3):186 192, 1993; Harada, et al., J. Oral Pathol. Med., 22(4):145 152, 1993; Stauber, et al., J. Immunol. Methods, 161(2):157 168, 1993; and Venkateswaran, et al., Hybridoma, 11(6) 729 739, 1992. These techniques can be used to produce antigen-reactive monoclonal antibodies, including antigen-specific IgG, and IgM monoclonal antibodies.

Any antibody or fragment thereof having affinity and specific for the bacteria of interest is within the scope of the invention provided herein. Immunomagnetic beads against Salmonella are provided in Vermunt et al. (J. Appl. Bact. 72:112, 1992). Immunomagnetic beads against Staphylococcus aureus are provided in Johne et al. (J. Clin. Microbiol. 27:1631, 1989). Immunomagnetic beads against Listeria are provided in Skjerve et al. (Appl. Env. Microbiol. 56:3478, 1990). Immunomagnetic beads against Escherichia coli are provided in Lund et al. (J. Clin. Microbiol. 29:2259, 1991).

Methods for attaching the target-specific binding moiety to the magnetic particle are known in the art. Coating magnetic particles with antibodies is well known in the art, see for example Harlow et al. (Antibodies, Cold Spring Harbor Laboratory, 1988), Hunter et al. (Immunoassays for Clinical Chemistry, pp. 147-162, eds., Churchill Livingston, Edinborough, 1983), and Stanley (Essentials in Immunology and Serology, Delmar, pp. 152-153, 2002). Such methodology can easily be modified by one of skill in the art to bind other types of target-specific binding moieties to the magnetic particles. Certain types of magnetic particles coated with a functional moiety are commercially available from Sigma-Aldrich (St. Louis, Mo.).

In certain embodiments, a buffer solution is added to the sample along with the magnetic beads. An exemplary buffer includes Tris(hydroximethyl)-aminomethane hydrochloride at a concentration of about 75 mM. It has been found that the buffer composition, mixing parameters (speed, type of mixing, such as rotation, shaking etc., and temperature) influence binding. It is important to maintain osmolality of the final solution (e.g., blood+buffer) to maintain high label efficiency. In certain embodiments, buffers used in methods of the invention are designed to prevent lysis of blood cells, facilitate efficient binding of targets with magnetic beads and to reduce formation of bead aggregates. It has been found that the buffer solution containing 300 mM NaCl, 75 mM Tris-HCl pH 8.0 and 0.1% Tween 20 meets these design goals.

Without being limited by any particular theory or mechanism of action, it is believed that sodium chloride is mainly responsible for maintaining osmolality of the solution and for the reduction of non-specific binding of magnetic bead through ionic interaction. Tris(hydroximethyl)-aminomethane hydrochloride is a well-established buffer compound frequently used in biology to maintain pH of a solution. It has been found that 75 mM concentration is beneficial and sufficient for high binding efficiency. Likewise, Tween 20 is widely used as a mild detergent to decrease nonspecific attachment due to hydrophobic interactions. Various assays use Tween 20 at concentrations ranging from 0.01% to 1%. The 0.1% concentration appears to be optimal for the efficient labeling of bacteria, while maintaining blood cells intact.

An alternative approach to achieve high binding efficiency while reducing time required for the binding step is to use static mixer, or other mixing devices that provide efficient mixing of viscous samples at high flow rates, such as at or around 5 mL/min. In one embodiment, the sample is mixed with binding buffer in ratio of, or about, 1:1, using a mixing interface connector. The diluted sample then flows through a mixing interface connector where it is mixed with target-specific nanoparticles. Additional mixing interface connectors providing mixing of sample and antigen-specific nanoparticles can be attached downstream to improve binding efficiency. The combined flow rate of the labeled sample is selected such that it is compatible with downstream processing.

After binding of the magnetic particles to the target in the mixture to form target/magnetic particle complexes, a magnetic field is applied to the mixture to capture the complexes on a surface. Components of the mixture that are not bound to magnetic particles will not be affected by the magnetic field and will remain free in the mixture. Methods and apparatuses for separating target/magnetic particle complexes from other components of a mixture are known in the art. For example, a steel mesh may be coupled to a magnet, a linear channel or channels may be configured with adjacent magnets, or quadrapole magnets with annular flow may be used. Other methods and apparatuses for separating target/magnetic particle complexes from other components of a mixture are shown in Rao et al. (U.S. Pat. No. 6,551,843), Liberti et al. (U.S. Pat. No. 5,622,831), Hatch et al. (U.S. Pat. No. 6,514,415), Benjamin et al. (U.S. Pat. No. 5,695,946), Liberti et al. (U.S. Pat. No. 5,186,827), Wang et al. (U.S. Pat. No. 5,541,072), Liberti et al. (U.S. Pat. No. 5,466,574), and Terstappen et al. (U.S. Pat. No. 6,623,983), the content of each of which is incorporated by reference herein in its entirety.

In certain embodiments, the magnetic capture is achieved at high efficiency by utilizing a flow-through capture cell with a number of strong rare earth bar magnets placed perpendicular to the flow of the sample. When using a flow chamber with flow path cross-section 0.5 mm×20 mm (h×w) and 7 bar NdFeB magnets, the flow rate could be as high as 5 mL/min or more, while achieving capture efficiency close to 100%.

In certain embodiments, the presence of magnetic particles that are not bound to target analytes and non-specific target entities on the surface that includes the target/magnetic particle complexes interferes with the ability to successfully detect the target of interest. The magnetic capture of the resulting mix, and close contact of magnetic particles with each other and labeled targets, result in the formation of aggregates that are hard to dispense and which might be resistant or inadequate for subsequent processing or analysis steps. Further, with addition of excess magnetic particles to the sample, a large number of particles may accumulate in the areas of high gradients, and thus a magnetically bound target analyte may likely be in the body of the accumulation of particles as opposed to the desired location adjacent the functionalized surface where specific binding may occur. Ignoring intra-bead forces (those forces associated with the magnetic field distribution of the individual beads and the forces these fields and associated gradients have on other beads), the beads may accumulate into large amorphous piles. Such intra-label forces do occur, and thus the aggregates of beads tend to exist in chains and long linear aggregates that are aligned with the ‘field lines’ of the magnetic trap pieces.

In certain embodiments, methods of the invention address this problem by applying alternating magnetic fields to the sample as it flows through the channel. In such embodiment, the flow chamber can be lined with second moieties specific to the target/magnetic particle complex. Alternating magnetic fields are then used to bring the target/magnetic particle complex in close proximity to the second moiety lined flow chamber and apart from other components in the sample.

The frequency of the alternating magnetic field is selected such that the free magnetic nanoparticles cannot transverse the whole distance between top and bottom of the flow cell before the direction of the magnetic field is changed, causing nanoparticles to move in the opposite direction. Therefore, a majority of free nanoparticles will not come into close contact with active surfaces of the flow cell and will be washed away by liquid flow. Labeled target, due to higher magnetic moment, have higher velocity in the magnetic field and will reach a surface of the flow cell before change of the magnetic field, thus coming into close contact with the surface. This, in turn, will result in a specific binding event and result in a specific capture of the target analyte in the sample (such as a bacterium or other rare cell) to the surface coated with a second moiety. Components of the mixture that are not bound to magnetic particles will not be affected by the magnetic field and will remain free in the mixture.

The second target-specific moiety may be the same or different from the first target-specific moiety. The second moiety may be attached to the surface of the flow channel by methods described above relating to attaching first target-specific moieties to magnetic particles.

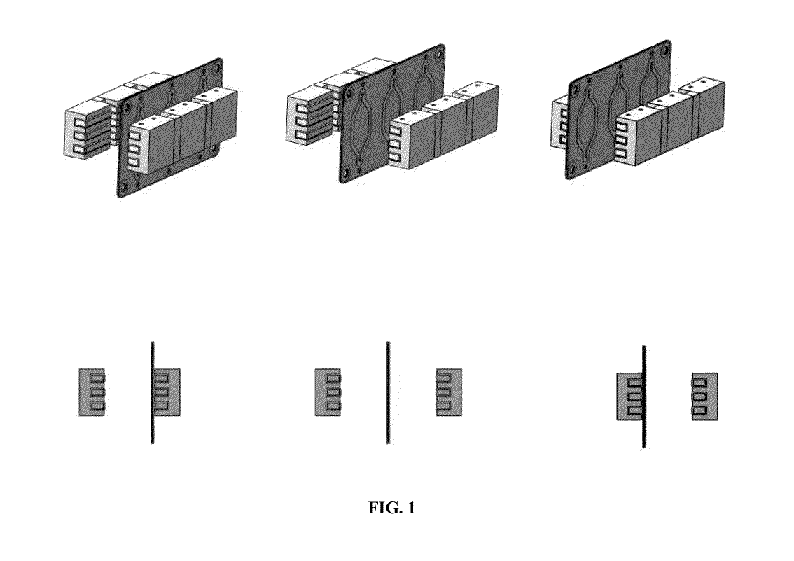

FIG. 1 provides one exemplary configuration of a flow cell and first and second sets of magnets for generating alternating magnetic fields. One of skill in the art will recognize that it is only an exemplary embodiment and that the separation can be achieved without using magnetic fields, as described in other embodiments throughout the application. This figure shows that the flow cell is positioned between the first and second sets of magnetics. Either movement of the flow cell or movement of the magnets brings the flow cell closer to one set of magnets and further from the other set of magnets. Subsequent movement brings the flow cell within proximity of the other set of magnets. Such movements generate alternating magnetic fields within the channels of the flow cell that are felt by the unbound magnetic particles and the target/magnetic particle complexes. In one embodiment, a flow cell may be about 15 mm wide and about 15 mm long, with a lead-in region and a lead-out section, and a height of about 0.5 mm (FIG. 1). A flow rate for such a cell may be about 100 μl/min, about 1 mL/min, about 10 mL/min, or from about 100 μL/min to about 10 mL/min or other ranges therein. A magnetic configuration may be an array of magnets, for example, an array of 7 bar magnets, or 5 bar magnets, or 3 bar magnets (FIG. 1). Magnets may be configured with alternating magnet poles facing one another, n-n, s-s, etc., with the pole face being normal to the array's rectangular face in this embodiment.

In a flowing system, successive encounters of unbound magnetic particles with a surface of a flow channel, without a resulting binding event, will allow the unbound magnetic particles to travel through the system and subsequently out of the cell. The cycling of the magnetic bar trap assemblies may be optimized based on the flow characteristics of the target(s) of interest. The expression of force on a magnetic moment and of terminal velocity for such target(s) is the following:

m dot(del B)=F Equation 1

vₜ=−F/(6*p*n*r) Equation 2

where n is the viscosity, r is the bead diameter, F is the vector force, B is the vector field, and m is the vector moment of the bead.

A characteristic transit time across the height of the cell may be established. An efficient frequency of the alternating magnetic attractors, such that many surface interactions may be established prior to the exit from the flow cell, may be established. In certain embodiments, the transit time can be substantially different for the target of interest versus the unbound magnetic particles, or non-specific bound non-target. In such an embodiment, the target can be ensured to interact with surface a maximal amount of times, while the unbound magnetic particles or non-target can interact a minimal number of times, or not at all.

Because of the magnetizing characteristics of the particles, the unbound magnetic particles may form aggregates, which may be in the form of linear chains or clumps. This may be the case at high concentrations of beads. At all concentrations, the unbound magnetic particles may exhibit spatial poison statistics, and there is some probability that there will be a neighboring bead close enough to be captured by the forces associated with the magnetic field of the beads themselves. By using alternating magnetic fields, methods of the invention break up these linear aggregates, particularly when the spatial gradient field from the trap magnets is shifted faster than the unbound magnetic particles can move mutually to reorient to the new distribution of trap gradient. Particles organized in chains, with N-S axis co-aligned, may quickly be subjected to an external field that produces particle moments with the N-S poles shifted by 90°, and may produce very strong intra-particle repulsive forces. Transverse motion of the trap magnets serves this purpose in concert with, or as a discrete step in addition to, the alternation of the trapping magnets from one surface to the other.

In the optimization of the cycling timing of the trap magnets, the flow characteristics of the cell may be considered along with the spatial distribution of the gradient of the trap magnets. Flow characteristics may dictate the transport of the magnetic materials from entrance to exit of the cell, so that parabolic flow, plug flow, or any particular flow characteristic may be considered to facilitate obtaining desired deposition patterns and desired interactions with the surfaces of interest.

In certain embodiments, it may be desirable in various applications to maximize the encounters of the target/magnetic particle complexes with the functionalized surface of the channel, to minimize interference with the unbound magnetic particles, and/or to minimize adhesion of the unbound magnetic particles and non-specific materials to the surface. It may be advantageous to produce an array of pipes, or tubes, through which the flow of the sample materials may flow. By way of example, a 125 mm×15 mm×0.5 mm cell volume may be filled with tubes, longitudinally aligned with the cell flow direction, such that there is a great increase in the functionalized surface area and a limitation on the number of unbound magnetic particles that may interact and impede in the encounter of the target with the surface. Planar structures may be used for this purpose, in which the cell volume is constructed with multiple layers of smaller flow channels such that the surface area is increased and the number of unbound magnetic particles available to impede the target on its way to the surface is decreased. In this embodiment, the general approach of cycling the trap magnets is similar to that described above, but variables such as time constants, amplitudes and gradient field distributions, for example, are optimized for the particular situation. Similarly, in the case of transverse trap motion for the breaking-up of aggregates, the general approach is similar to that described above.

It may be desirable to shield the portion of the sample flow outside the trap cell from fringing magnetic field so that magnetic material does not have the opportunity to self-aggregate prior to entering the strong field and gradient zone of the trap. The magnetic materials and labeled target may also be trapped in flow tubes and other fluidic structures through magnetic forces in undesired areas. Shielding can be accomplished by the appropriate design of the trap magnets, for example, by managing the ‘return path’ of the field, and/or by using high permeability materials to capture and channel the field to minimize fringing field exposure.

The above described type of magnetic separation produces efficient capture of a target and the removal of a majority of the remaining components of a sample mixture. However, such a process may produce a sample that contains a percent of magnetic particles that are not bound to targets, as well as non-specific target entities. Non-specific target entities may for example be bound at a much lower efficiency, for example 1% of the surface area, while a target of interest might be loaded at 50% or nearly 100% of the available surface area or available antigenic cites. However, even 1% loading may be sufficient to impart force necessary for trapping in a magnetic gradient flow cell or sample chamber.

The presence of magnetic particles that are not bound to targets and non-specific target entities on the surface that includes the target/magnetic particle complexes may interfere with the ability to successfully detect the target of interest. The magnetic capture of the resulting mix, and close contact of magnetic particles with each other and bound targets, result in the formation of aggregate that is hard to dispense and which might be resistant or inadequate for subsequent processing or analysis steps. In order to remove magnetic particles that are not bound to targets and non-specific target entities, methods of the invention may further involve washing the surface with a wash solution that reduces particle aggregation, thereby isolating target/magnetic particle complexes from the magnetic particles that are not bound to targets and non-specific target entities. The wash solution minimizes the formation of the aggregates.

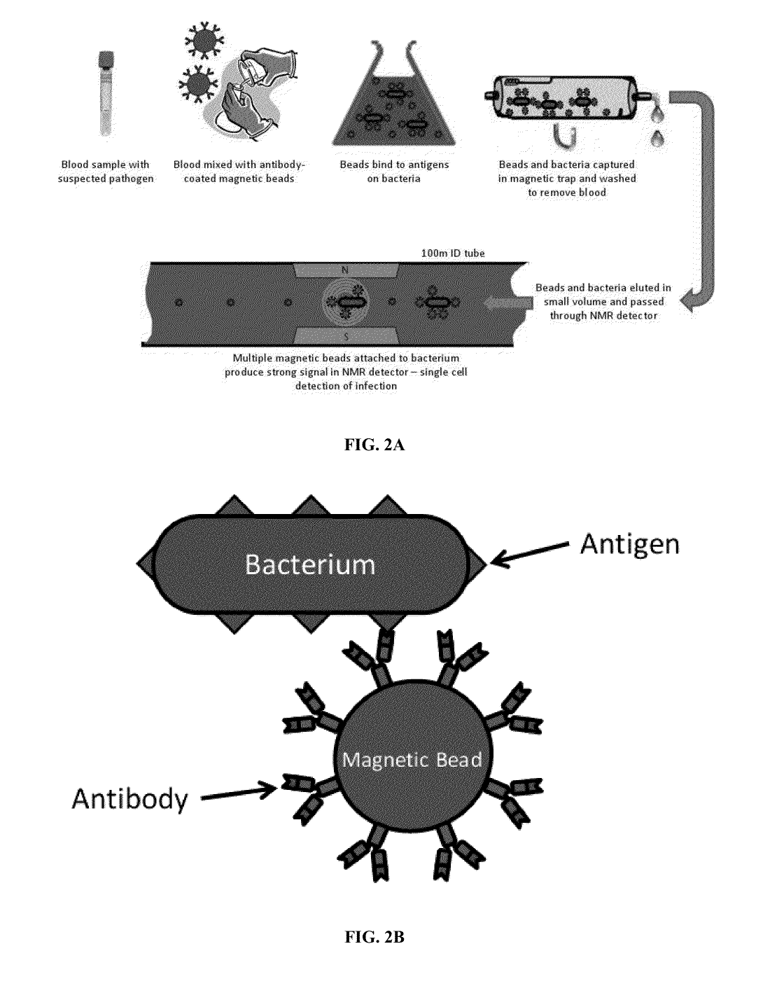

FIG. 2A provides an exemplary process chart for implementation of methods of the invention for separation of bacteria from blood. Sample is collected in sodium heparin tube by venipuncture, acceptable sample volume is 1-10 mL. Superparamagnetic particles having target-specific binding moieties are added to the sample, followed by incubation on a shaking incubator at 37° C. for 30-120 min. FIG. 2B provides an exemplary view of the target/magnetic particle complex, illustrating the binding between the two species.

Methods of the invention may use any wash solution that imparts a net negative charge to the magnetic particle that is not sufficient to disrupt interaction between the target-specific moiety of the magnetic particle and the target. Without being limited by any particular theory or mechanism of action, it is believed that attachment of the negatively charged molecules in the wash solution to magnetic particles provides net negative charge to the particles and facilitates dispersal of non-specifically aggregated particles. At the same time, the net negative charge is not sufficient to disrupt strong interaction between the target-specific moiety of the magnetic particle and the target (e.g., an antibody-antigen interaction). Exemplary solutions include heparin, Tris-HCl, Tris-borate-EDTA (TBE), Tris-acetate-EDTA (TAE), Tris-cacodylate, HEPES (4-(2-hydroxyethyl)-1-piperazineethanesulphonic acid), PBS (phosphate buffered saline), PIPES (piperazine-N,N′-bis(2-ethanesulfonic acid), MES (2-N-morpholino)ethanesulfonic acid), Tricine (N-(Tri(hydroximethyl)methyl)glycine), and similar buffering agents. In certain embodiments, only a single wash cycle is performed. In other embodiments, more than one wash cycle is performed.

In particular embodiments, the wash solution includes heparin. For embodiments in which the body fluid sample is blood, the heparin also reduces probability of clotting of blood components after magnetic capture. The bound targets are washed with heparin-containing buffer 1-3 times to remove blood components and to reduce formation of aggregates.

Once the target/magnetic particle complexes are isolated, the target may be analyzed by a multitude of existing technologies, such as miniature NMR, Polymerase Chain Reaction (PCR), mass spectrometry, fluorescent labeling and visualization using microscopic observation, fluorescent in situ hybridization (FISH), growth-based antibiotic sensitivity tests, and variety of other methods that may be conducted with purified target without significant contamination from other sample components. In a preferred embodiment, the target is analyzed in order to identify the genes expressed by the target.

In certain embodiments, after targets have been obtained from the sample, it is preferable to lyse targets in order to isolate nucleic acids that can be found within the targets. Such targets may be a virus, a microorganism (e.g., bacteria or fungi) or cells (such as normal or abnormal cells, such as cancerous cells). Once the target/magnetic particle complexes have been captured, the process of lysis can be initiated. It will be appreciated by one skilled in the art that depending on the type of technique of analysis to be performed on the analyte, lysing can be applied to the method, or the process of lysing can be omitted. Lysing of the target/magnetic particle complexes occurs without disturbing the binding between the target and the magnetic particle, or that the target/magnetic particle complex is maintained during the lysing step.

Conducting lysis without the pre-separation step of the target and magnetic particle allows for more efficacious collection of analytes contained within the target. For instance, if the analyte of interest is a bacterially-derived nucleic acid, some analyte may be lost when bacteria separated from the magnetic particles are inadvertently lost. With the disclosed methods, the bacteria are still bound to the particles and captured on a surface as lysis occurs. Accordingly, there is a concentrated sample to work with during the lysing step. In addition, the disclosed methods enable recovery of a specific analyte in a relatively small collection volume. This is especially useful when the analyte of interest is a nucleic acid or something that is similarly present in only very small quantities. For example, one could begin with a blood sample of 2 mL and use the described methods to concentrate a desired pathogen from the blood onto an appropriate surface. With the pathogen captured, one could decant the blood, add 0.3 mL of a suitable buffer, and subsequently perform the lysis step. In alternative embodiments, the lysis process is conducted after separation of the target(s) from other components of the sample.

Lysis can be performed using any means known in the art. For example, lysis could be performed using a lysis buffer. Any type of lysis buffer is suitable for use with the disclosed methods; selection of the specific buffer may depend on the subsequent analysis of the cell lysate. Buffer selection is within the general skill of the art and can be determined empirically. Generally, lysis buffers contain tris-HCL, EDTA, EGTA, SDS, deoxycholate, Triton X, and/or NP-40. In some cases the buffer may also contain NaCl (150 mM). In certain aspects, the lysis buffer is a chaotropic solution.

Lysis can also be achieved through sonication. In this method, the captured target/magnetic particle complexes are exposed to ultrasonic waves to achieve lysis of any target (bacteria, cells, virus, fungi, etc.) associated with the magnetic particles so that any analytes of interest contained therein are released. In some embodiments, the analyte of interest can include a nucleic acid.

The methods described herein can be used in accordance with any sonication device, which are well-known in the art. In certain embodiments, the sonication device is a VIBRA-CELLVCX 750 Sonicator (sonicator, commercially available by Sonics & Materials, Inc.). Generally, the probe of the sonicator is placed into the liquid containing the targets to be lysed. Electrical energy from a power source is transmitted to a piezoelectric transducer within the sonicator converter, where it is changed to mechanical vibrations. The longitudinal vibrations from the converter are intensified by the probe, creating pressure waves in the liquid. These in turn produce microscopic bubbles, which expand during the negative pressure excursion and implode violently during the positive excursion. This phenomenon, referred to as cavitation, creates millions of shock waves and releases high levels of energy into the liquid, thereby lysing the target. In another embodiment, the sonication transducer may be brought in contact with a chamber holding captured complexes by way of a structural interface. The sonication transducer vibrates the structural interface such that lysis is achieved. In either method, the appropriate intensity and period of sonication can be determined empirically by those skilled in the art.

After the process of lysis, the lysate contains the analyte, and thereby the contents of the lysed target can then be eluted. In certain aspects, the analyte contains nucleic acids of interest associated with a particular bacteria present in the starting sample. With the process of elution, the analytes are removed from the magnetic particles, wherein an eluent could be employed. Analytes may include, without limitation, nucleic acids, proteins, organelles, and other components found within the target of interest.

The contents of the target are purified by standard methods of nucleic acid purification. Cellular extracts can be subjected to other steps to drive nucleic acid isolation toward completion by, e.g., differential precipitation, column chromatography, extraction with organic solvents and the like. Extracts then may be further treated, for example, by filtration and/or centrifugation and/or with chaotropic salts such as guanidinium isothiocyanate or urea or with organic solvents such as phenol and/or HCCl₃ to denature any contaminating and potentially interfering proteins. The nucleic acid can also be resuspended in a hydrating solution, such as an aqueous buffer. The nucleic acid can be suspended in, for example, water, Tris buffers, or other buffers.

Methods of detecting levels of gene products (e.g., RNA or protein) are known in the art. Commonly used methods known in the art for the quantification of mRNA expression in a sample include northern blotting and in situ hybridization (Parker & Barnes, Methods in Molecular Biology 106:247 283 (1999), the contents of which are incorporated by reference herein in their entirety); RNAse protection assays (Hod, Biotechniques 13:852 854 (1992), the contents of which are incorporated by reference herein in their entirety); and PCR-based methods, such as reverse transcription polymerase chain reaction (RT-PCR) (Weis et al., Trends in Genetics 8:263 264 (1992), the contents of which are incorporated by reference herein in their entirety). Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

In some embodiments, messenger RNA transcripts are converted into its complementary DNA sequence for analysis. Methods of the invention provide for converting RNA to cDNA. RNA can be converted to cDNA using any method known in the art. Generally, RNA to cDNA conversion steps include purifying messenger RNA (mRNA) using poly-A selection, performing reverse transcriptase and performing oligonucleotide-primed synthesis of cDNA.

RNA is usually converted into first strand cDNA enzymatically by reverse transcriptase (RT), a RNA-dependent DNA polymerase. Exemplary reverse transcriptase (RT) includes, but not limited to, the Moloney murine leukemia virus (M-MLV) RT as described in U.S. Pat. No. 4,943,531, a mutant form of M-MLV-RT lacking Rnase H activity as described in U.S. Pat. No. 5,405,776, human T-cell leukemia virus type I (HTLV-I) RT, bovine leukemia virus (BLV) RT, Rous sarcoma virus (RSV) RT, Avian Myeloblastosis Virus (AMV) RT and human immunodeficiency virus (HIV) RT. RTs suitable for this purpose may also be extracted from their natural hosts. Alternatively, RTs can be obtained commercially or isolated from host cells that express high levels of recombinant forms of the enzymes by methods known to those of skill in the art. The particular manner of obtaining the reverse transcriptase may be chosen based on factors such as convenience, cost, availability and the like.

Reverse transcriptase can extend the free end of an oligonucleotide (or a primer) that forms a stable base pairing with the target RNA molecule. Under most conditions, RT enzymes can produce cDNA molecules without the supply of exogenous primers. Alternatively, exogenous primers may be used. Two types of exogenous primers, random primers and specific primers, may be added to the reaction to facilitate the cDNA synthesis. Random primers, which have defined length but no defined sequence, can be used to prime the conversion of RNA to cDNA without discrimination of RNA species. The length of random primers is usually between 2 and 25 nucleotides (nt), but more often between 5 and 10 nt. The most commonly used is the random hexamers (6 nt). Specific primers, which have defined length and defined nucleotide sequence, may also be used to synthesize cDNA from a defined sub-population of RNA. An example of such specific primers is the oligo dT primers. Oligo dTs primers are a short sequence of deoxy-thymine nucleotides that are tagged as complementary primers which bind to the poly-A tail providing a free 3′-OH end, a characteristic of most messenger RNA in cells, which can be extended by reverse transcriptase to create the complementary DNA strand. The length of the oligo dT primers may be between 10 to 40 nt, between 15 and 25 nt, or about 18 nt. Another example of a specific primer is the gene-specific primer. A gene-specific primer may have a sequence complementary to that of a distinct RNA. Preferably, the gene-specific primer has a sequence substantially complementary to that of a distinct RNA. In some embodiments, a gene-specific primer primes the synthesis of cDNA from one unique sequence within a RNA molecule that corresponds to one single gene.

Exogenous primers can be synthesized according to conventional oligonucleotide chemistry methods, in which the nucleotide units may be: (A) solely nucleotides found in naturally occurring DNA and RNA, e.g., adenine, cytosine, guanine, thymine and uracil; or (B) solely nucleotide analogs that are capable of base pairing under hybridization conditions in the course of DNA synthesis such that they function as the nucleotides described in (A), e.g., inosine, xanthine, hypoxanthine, 1,2-diaminopurine and the like; or (C) any combination of the nucleotides described in both (A) and (B).

The buffer necessary for first strand cDNA synthesis may be purchased commercially from various sources, such as SuperArray, Promega, Invitrogen, Clontech, Amersham. These buffers have a pH ranging from 6 to 9, with 10-200 mM of Tris-HCl or HEPES. Other salts may include NaCl, KCl, MgCl₂, Mg (OAc)₂, MnCl₂, Mn(OAc)₂ etc., at concentrations ranging from 1-200 mM. Additional reagents such as reducing agents (DTT), detergents (TritonX-100), albumin and the like may be supplemented in the buffer. Chemical compound or polymers, such as DMSO, poly-lysine, betaine, and the like, may be added to the buffer to prevent RNA from forming secondary structures. Depending on the particular nature of the assay, a combination of the above mentioned reagents might be chosen to limit endogenous priming during reverse transcription.

Deoxyribonucleoside triphosphates (dNTPs) necessary for first strand cDNA synthesis through reverse transcription of RNAs may be purchased commercially from various sources, such as SuperArray, Promega, Invitrogen, Clontech, Amersham. In the reaction, dNTPs may include nucleotides that are commonly found in DNA, e.g. dATP, dGTP, dCTP dTTP and dUTP; analogs of above mentioned nucleotides that are less frequently found in nature, such as those with ribose moieties like inosine, xanthine, hypoxanthine; and combinations of the nucleotides commonly found in DNA and their analogs. The combination of nucleotide analogs may be helpful separating the newly synthesized DNA from the genomic DNA that may co-purify with the total RNA. Derivatives of inosine are an example commonly used for this purpose. Newly synthesized dITP-containing DNA has a lower melting temperature than the corresponding natural DNA, such as genomic DNA (Auer et al. Nucleic Acids Res. 1996; 24:5021-5025, U.S. Pat. No. 5,618,703), and (Levy D D and Teebor G W. Nuc. Acids Res. 1991; 19(12):3337-3343; Warren R A. Annu. Rev. Microbiol. 1980; 34:137-158). Another unconventional nucleotide affecting the Tm of the newly synthesized DNA product is hydroxymethyl dUTP (HmdUTP), which occurs naturally in phage SP01 genomic DNA in place of dTTP. The melting temperature of HmdUTP-containing DNA is 10° C. lower than normal DNA. (Levy D D and Teebor G W. Nuc. Acids Res. 1991; 19(12):3337-3343).

The resulting single stranded cDNA is converted into a double stranded DNA with the help of a DNA polymerase. However, for DNA polymerase to synthesize a complementary strand a free 3′-OH end is needed. This is provided by the single stranded cDNA itself by generating a hairpin loop at the 3′ end by coiling on itself. The polymerase extends the 3′-OH end and later the loop at 3′ end is opened by the scissoring action of an S₁ nuclease.

In addition to the above described techniques for conversion of RNA into double stranded RNA, other methods of conversion are described in detail in Klickstein et al., 2001. Conversion of mRNA into Double-Stranded cDNA. Current Protocols in Molecular Biology. 29:5.5.1-5.5.14, the contents of which is incorporated by reference in its entirety. Klickstein describes two protocols for converting RNA into double stranded cDNA. One protocol describes a method for making blunt-ended cDNA that can then be ligated to linkers for subsequent cloning into a unique restriction site, and the other the other protocol is a variation that requires fewer enzymatic manipulations and allows construction of directional cDNA libraries.

In addition, kits are commercially available for conversion of RNA to cDNA from, for example, Illumina (San Diego, Calif.), Life Technologies (Foster City, Calif.), and Qiagen, (Valencia, Calif.). User Guides that describe in detail the protocol(s) to be followed are usually included in all these kits.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure.

The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR (gene expression assay, commercially available by Life Technologies company) typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR (gene expression assay, commercially available by Life Technologies company) can be performed using commercially available equipment, such as, for example, ABI PRISM 7700 SEQUENCE DETECTION SYSTEM (sequence detection system, commercially available from Perkin-Elmer-Applied Biosystems, Foster City, Calif., USA), or Lightcycler (Roche Molecular Biochemicals, Mannheim, Germany). In certain embodiments, the 5′ nuclease procedure is run on a real-time quantitative PCR device such as the ABI PRISM 7700 SEQUENCE DETECTION SYSTEM (sequence detection system, commercially available from Perkin-Elmer-Applied Biosystems, Foster City, Calif., USA). The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Cₜ).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin. For performing analysis on pre-implantation embryos and oocytes, Chuk is a gene that is used for normalization.

A more recent variation of the RT-PCR technique is the real time quantitative PCR, which measures PCR product accumulation through a dual-labeled fluorigenic probe (i.e., TAQMAN (gene expression assay, commercially available by Life Technologies company) probe). Real time PCR is compatible both with quantitative competitive PCR, in which internal competitor for each target sequence is used for normalization, and with quantitative comparative PCR using a normalization gene contained within the sample, or a housekeeping gene for RT-PCR. For further details see, e.g. Held et al., Genome Research 6:986 994 (1996), the contents of which are incorporated by reference herein in their entirety.

In another embodiment, a MASSARRAY (DNA mass array, commercially available by Sequenom, Inc.) based gene expression profiling method is used to measure gene expression. In the MASSARRAY (DNA mass array, commercially available by Sequenom, Inc.) based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059 3064 (2003).

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BEADARRAY technology (microarray plateform, commercially available by Illumina Inc.) (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269), the content of which is incorporated by reference herein in its entirety.

In a specific embodiment of the microarray technique, PCR amplified inserts of cDNA clones are applied to a substrate in a dense array, for example, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996), the contents of which are incorporated by reference herein in their entirety). Microarray analysis can be performed by commercially available equipment, following manufacturer's protocols, such as by using the Affymetrix GenChip technology, or Incyte's microarray technology.

Alternatively, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484 487 (1995); and Velculescu et al., Cell 88:243 51 (1997, the contents of each of which are incorporated by reference herein in their entirety).

In other embodiments Massively Parallel Signature Sequencing (MPSS) is used to measure gene expression. This method, described by Brenner et al., Nature Biotechnology 18:630 634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunohistochemistry methods are also suitable for detecting the expression levels of the gene products of the present invention. Thus, antibodies (monoclonal or polyclonal) or antisera, such as polyclonal antisera, specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See for example U.S. Pat. Nos. 6,925,389; 6,989,100; and 6,890,763 for further guidance, each of which is incorporated by reference herein in their entirety.

Methods of the invention also provide for generating a cDNA library from the total RNA extracted from the pathogen. Methods known in the art for cDNA library generation are suitable for use in methods of the invention, for example, use of a olgio-(DT) primed and directional cloned strategy for generating cDNA libraries. Similarly, methods for cDNA library screening to identify cDNA library clones representing genes of interest are also widely known, and include, for example, homology screening and DNA/protein interaction screens, and various forms of expression screening such as antibody-based immunoscreening, protein/protein interaction screening, and screenings based on functional assays. Methods and reagents for library construction and screening are available in a variety of sources, including but not limited to, Ausubel et al. (eds.), Current Protocols in Molecular Biology, Vol. 1-4, John Wiley & Sons, Inc., New York (1994) and Sambrook et al. (eds.), Molecular Cloning: A Laboratory Manual, Second Edition, Vol. 1-3, Cold Spring Harbor Laboratory Press, NY (1989).

In certain aspects, it may be preferable to detect the presence of the target prior to determining the expression profile of the target. Because the assay is capable of detecting a single target cell from a large volume of sample, it can be beneficial to know how much target there is for prior to expression analysis. For example, if there is only a small amount of target present, one may choose a more sensitive assay to determine the gene expression profile. In such embodiments, the following techniques may be used to determine the presence and/or amount of captured target within the sample that can be analyzed for gene expression.

In one embodiment, captured bacteria is removed from the magnetic particles to which they are bound and the processed sample is mixed with fluorescent labeled antibodies specific to the bacteria or fluorescent Gram stain. After incubation, the reaction mixture is filtered through 0.2 μm to 1.0 μm filter to capture labeled bacteria while allowing majority of free beads and fluorescent labels to pass through the filter. Bacteria is visualized on the filter using microscopic techniques, e.g. direct microscopic observation, laser scanning or other automated methods of image capture. The presence of bacteria is detected through image analysis. After the positive detection by visual techniques, the bacteria can be further characterized using PCR or genomic methods.

Detection of bacteria of interest using NMR may be accomplished as follows. In the use of NMR as a detection methodology, in which a sample is delivered to a detector coil centered in a magnet, the target of interest, such as a magnetically labeled bacterium, may be delivered by a fluid medium, such as a fluid substantially composed of water. In such a case, the magnetically labeled target may go from a region of very low magnetic field to a region of high magnetic field, for example, a field produced by an about 1 to about 2 Tesla magnet. In this manner, the sample may traverse a magnetic gradient, on the way into the magnet and on the way out of the magnet. As may be seen via equations 1 and 2 above, the target may experience a force pulling into the magnet in the direction of sample flow on the way into the magnet, and a force into the magnet in the opposite direction of flow on the way out of the magnet. The target may experience a retaining force trapping the target in the magnet if flow is not sufficient to overcome the gradient force.

Magnetic fields on a path into a magnet may be non-uniform in the transverse direction with respect to the flow into the magnet. As such, there may be a transverse force that pulls targets to the side of a container or a conduit that provides the sample flow into the magnet. Generally, the time it takes a target to reach the wall of a conduit is associated with the terminal velocity and is lower with increasing viscosity. The terminal velocity is associated with the drag force, which may be indicative of creep flow in certain cases. In general, it may be advantageous to have a high viscosity to provide a higher drag force such that a target will tend to be carried with the fluid flow through the magnet without being trapped in the magnet or against the conduit walls.

Newtonian fluids have a flow characteristic in a conduit, such as a round pipe, for example, that is parabolic, such that the flow velocity is zero at the wall, and maximal at the center, and having a parabolic characteristic radius. The velocity decreases in a direction toward the walls, and it is easier to magnetically trap targets near the walls, either with transverse gradients force on the target toward the conduit wall, or in longitudinal gradients sufficient to prevent target flow in the pipe at any position. In order to provide favorable fluid drag force to keep the samples from being trapped in the conduit, it may be advantageous to have a plug flow condition, wherein the fluid velocity is substantially uniform as a function of radial position in the conduit.

When NMR detection is employed in connection with a flowing sample, the detection may be based on a perturbation of the NMR water signal caused by a magnetically labeled target (Sillerud et al., JMR (Journal of Magnetic Resonance), vol. 181, 2006). In such a case, the sample may be excited at time 0, and after some delay, such as about 50 ms or about 100 ms, an acceptable measurement (based on a detected NMR signal) may be produced. Alternatively, such a measurement may be produced immediately after excitation, with the detection continuing for some duration, such as about 50 ms or about 100 ms. It may be advantageous to detect the NMR signal for substantially longer time durations after the excitation.

By way of example, the detection of the NMR signal may continue for a period of about 2 seconds in order to record spectral information at high-resolution. In the case of parabolic or Newtonian flow, the perturbation excited at time 0 is typically smeared because the water around the perturbation source travels at different velocity, depending on radial position in the conduit. In addition, spectral information may be lost due to the smearing or mixing effects of the differential motion of the sample fluid during signal detection. When carrying out an NMR detection application involving a flowing fluid sample, it may be advantageous to provide plug-like sample flow to facilitate desirable NMR contrast and/or desirable NMR signal detection.

Differential motion within a flowing Newtonian fluid may have deleterious effects in certain situations, such as a situation in which spatially localized NMR detection is desired, as in magnetic resonance imaging. In one example, a magnetic object, such as a magnetically labeled bacterium, is flowed through the NMR detector and its presence and location are detected using MRI techniques. The detection may be possible due to the magnetic field of the magnetic object, since this field perturbs the magnetic field of the fluid in the vicinity of the magnetic object. The detection of the magnetic object is improved if the fluid near the object remains near the object. Under these conditions, the magnetic perturbation may be allowed to act longer on any given volume element of the fluid, and the volume elements of the fluid so affected will remain in close spatial proximity. Such a stronger, more localized magnetic perturbation will be more readily detected using NMR or MRI techniques.

If a Newtonian fluid is used to carry the magnetic objects through the detector, the velocity of the fluid volume elements will depend on radial position in the fluid conduit. In such a case, the fluid near a magnetic object will not remain near the magnetic object as the object flows through the detector. The effect of the magnetic perturbation of the object on the surrounding fluid may be smeared out in space, and the strength of the perturbation on any one fluid volume element may be reduced because that element does not stay within range of the perturbation. The weaker, less-well-localized perturbation in the sample fluid may be undetectable using NMR or MRI techniques.

Certain liquids, or mixtures of liquids, exhibit non-parabolic flow profiles in circular conduits. Such fluids may exhibit non-Newtonian flow profiles in other conduit shapes. The use of such a fluid may prove advantageous as the detection fluid in an application employing an NMR-based detection device. Any such advantageous effect may be attributable to high viscosity of the fluid, a plug-like flow profile associated with the fluid, and/or other characteristic(s) attributed to the fluid that facilitate detection. As an example, a shear-thinning fluid of high viscosity may exhibit a flow velocity profile that is substantially uniform across the central regions of the conduit cross-section. The velocity profile of such a fluid may transition to a zero or very low value near or at the walls of the conduit, and this transition region may be confined to a very thin layer near the wall.

Not all fluids, or all fluid mixtures, are compatible with the NMR detection methodology. In one example, a mixture of glycerol and water can provide high viscosity, but the NMR measurement is degraded because separate NMR signals are detected from the water and glycerol molecules making up the mixture. This can undermine the sensitivity of the NMR detector. In another example, the non-water component of the fluid mixture can be chosen to have no NMR signal, which may be achieved by using a perdeuterated fluid component, for example, or using a perfluorinated fluid component. This approach may suffer from the loss of signal intensity since a portion of the fluid in the detection coil does not produce a signal.

Another approach may be to use a secondary fluid component that constitutes only a small fraction of the total fluid mixture. Such a low-concentration secondary fluid component can produce an NMR signal that is of negligible intensity when compared to the signal from the main component of the fluid, which may be water. It may be advantageous to use a low-concentration secondary fluid component that does not produce an NMR signal in the detector. For example, a perfluorinated or perdeuterated secondary fluid component may be used.

The fluid mixture used in the NMR detector may include one, two, or more than two secondary components in addition to the main fluid component. The fluid components employed may act in concert to produce the desired fluid flow characteristics, such as high-viscosity and/or plug flow. The fluid components may be useful for providing fluid characteristics that are advantageous for the performance of the NMR detector, for example by providing NMR relaxation times that allow faster operation or higher signal intensities.

A non-Newtonian fluid may provide additional advantages for the detection of objects by NMR or MRI techniques. As one example, the objects being detected may all have substantially the same velocity as they go through the detection coil. This characteristic velocity may allow simpler or more robust algorithms for the analysis of the detection data. As another example, the objects being detected may have fixed, known, and uniform velocity. This may prove advantageous in devices where the position of the detected object at later times is needed, such as in a device that has a sequestration chamber or secondary detection chamber down-stream from the NMR or MRI detection coil, for example.

In an exemplary embodiment, sample delivery into and out of a 1.7 T cylindrical magnet using a fluid delivery medium containing 0.1% to 0.5% Xanthan gum in water was successfully achieved. Such delivery is suitable to provide substantially plug-like flow, high viscosity, such as from about 10 cP to about 3000 cP, and good NMR contrast in relation to water. Xanthan gum acts as a non-Newtonian fluid, having characteristics of a non-Newtonian fluid that are well known in the art, and does not compromise NMR signal characteristics desirable for good detection in a desirable mode of operation.

In certain embodiments, methods of the invention are useful for direct detection of pathogens from blood. Such a process for a bacteria pathogen is described here. Sample is collected in sodium heparin tube by venipuncture, acceptable sample volume is about 1 mL to 10 mL. Sample is diluted with binding buffer and superparamagnetic particles having target-specific binding moieties are added to the sample, followed by incubation on a shaking incubator at 37° C. for about 30 min to 120 min. Alternative mixing methods can also be used. In a particular embodiment, sample is pumped through a static mixer, such that reaction buffer and magnetic beads are added to the sample as the sample is pumped through the mixer. This process allows for efficient integration of all components into a single fluidic part, avoids moving parts and separate incubation vessels and reduces incubation time.

Capture of the labeled targets allows for the removal of blood components and reduction of sample volume from 30 mL to 5 mL. The capture is performed in a variety of magnet/flow configurations. In certain embodiments, methods include capture in a sample tube on a shaking platform or capture in a flow-through device at flow rate of 5 mL/min, resulting in total capture time of 6 min.

After capture, the sample is washed with wash buffer including heparin to remove blood components and free beads. The composition of the wash buffer is optimized to reduce aggregation of free beads, while maintaining the integrity of the bead/target or target/magnetic particle complexes. The detection method is based on a miniature NMR detector tuned to the magnetic resonance of water. When the sample is magnetically homogenous (no bound targets), the NMR signal from water is clearly detectable and strong. The presence of magnetic material in the detector coil disturbs the magnetic field, resulting in reduction in water signal. One of the primary benefits of this detection method is that there is no magnetic background in biological samples which significantly reduces the requirements for stringency of sample processing. In addition, since the detected signal is generated by water, there is a built-in signal amplification which allows for the detection of a single labeled bacterium.

This method provides for isolation and detection of as low as or even lower than 1 CFU/mL of bacteria in a blood sample. Once isolated and detected, the bacteria can be examined for identification of genes expressed in the bacteria.

In a preferred embodiment, the target is analyzed in order to identify genes or fragments of nucleic acids expressed or contained within the target. DNA sequencing is well known in the art and has been described in detail in the preceding sections. In this embodiment, captured bacteria are lysed without first separating the bacteria from the magnetic particles. The lysate or analyte is then eluted from the magnetic particles and DNA contained within the lysate/analyte is bound to DNA extraction resin. After washing of the resin, the bacterial DNA is eluted and used in quantitative RT-PCR to detect the presence of a specific species, and/or, subclasses of bacteria, through nucleic acid identification or nucleic acid-nucleic acid comparison, such as DNA-DNA comparison, by means well known in the art. See for example IJSEM, Rademaker, et al., March 2000, 50:2, p. 665-677, which is incorporated by reference, and describes using repetitive sequence based (rep)-PCR and AFLP genomic fingerprinting to DNA-DNA hybridization studies to identify and classify known strains of microorganisms. Comparison analysis allows for the determination and identification of a known bacterium, pathogen, microorganism, microbe, prokaryote, or virus.

In another preferred embodiment, after the lysate/analyte is eluted, genetic variations or mutations within a species are identified by nucleic acid-nucleic acid comparison. It is well known in the art that pathogens, microorganisms, viruses, and bacteria mutate or can change genetically. Identifying these mutations is desired for further identification and determination of genetic closeness, along with determination of genetic distances between species. See for example J. Clin. Microbiol. December 2003 vol. 41 no. 12 5456-5465, which discloses guidelines for classification by using sequences of rRNA and protein-coding genes.

Furthermore, in another preferred embodiment, after the lysate/analyte is eluted nucleic acid quantification can be completed by means known in the art, as described in Appl. Environ. Microbiol. January 2002 vol. 68 no. 1 245-253, which discloses utilizing PCR and statistics to quantify bacterial populations. Quantification allows for not only identification, but allows for determination of the amount of bacterial or pathogenic species present in the sample. In particular applications, quantification is needed to determine the level of infection or contamination within a sample, and therefore, identification alone would be insufficient.

In another preferred embodiment, the captured target, such as a virus, could be lysed without first being eluted from the magnetic particles. The lysate/analyte is then eluted from the magnetic particles and DNA contained within the lysate/analyte is bound to DNA extraction resin. After washing of the resin, the DNA is eluted and used in quantitative RT-PCR to identify and quantify the presence of a virus in a sample. By known methods in the art, such as DNA microarrays (Wang et. al., PLOS, Nov. 17, 2003, DOI: 10.1371/journal.pbio.0000002, incorporated by reference) viral infections can be identified to determine which viruses are present in the sample. Furthermore, this embodiment can be used to identify whether the sample contains a pathogen or virus associated with a known infectious disease. In addition, the present invention can be employed for the means of classification of bacterial or viral species, including microorganisms and pathogens. This embodiment would be preferred for creating and establishing a classification system of the particular microorganisms or pathogens that are present in a particular group of samples.

In another preferred embodiment, after elution and sequencing of the nucleic acid as described above, mutations within a classification or species can be detected and identified. Known methods in the art may be employed, for example, J. Clin. Microbiol. January 1995 vol. 33 no. 1 248-250 (utilizes mismatch amplification mutation assay-multiplex PCR); PNAS Jul. 1, 1984 vol. 81 no. 13 4154-4158 (utilizes electrophoresis for the identification of mutations); and PNAS May 23, 2006 vol. 103 no. 21 8107-8112 (utilizes PCR amplification and capillary sequencing) are incorporated by reference. By identification of nucleic acids and particular genes, the level and degree of mutations of a microorganism, pathogen, virus, or bacteria can be determined if present in the sample. Identification of mutations within a species or classification allows for cataloging the changes in order to map evolutionary processes. This embodiment would allow for collection of data for understanding how mutation, phenotypic variation, and natural selection shape evolutionary processes. This method would allow for determination of whether a mutation within a species or classification exists, and the genetic closeness or similarities to other known mutations. For example, see Lancet, vol. 361, Issue 9371, 24 May 2003, pages 1779-1785, which is incorporated by reference and discloses genome sequence analysis to indicate new strain differences in viruses to identify geographical origins and provide insights into vaccine developments. With detection and identification of mutations or varying differences, the point of origin of a microorganism can be determined, which can give information about the point of contamination, infection, or integration. See J. Clin. Microbiol. June 1999 vol. 37 no. 6 1661-1669, which describes employing electrophoresis in subtyping and strain classification. Determining the location based upon strain development or mutation can lead to information about point source contamination or point source infection.

In another embodiment, gene expression profiling can be employed to measure the activity of genes within the target. Gene expression profiling simultaneously compares the expression levels of numerous genes within a sample, or between two or more sample types. Identification of genes expressed by a pathogen during active blood borne infection is beneficial in identifying agents in antimicrobial therapy. See Wolk, et al., Eur. J. Immunol., 36: 1309-1323. doi: 10.1002/eji.200535503, which discloses identify agents that influence gene expression in development of antimicrobial therapy.

In another embodiment, a gene expression profile can be created to determine the genes expressed by the pathogen. The gene expression profiles of pathogens allow for identification of transcripts encoding for surface antigens, which can be used for antibody generation. See Molecular Microbiology, 44: 9-19. doi: 10.1046/j.1365-2958.2002.02813, which discloses identification of Mycobacterium tuberculosis genes that were determined to be expressed utilizing RT-PCR to identify variable surface antigens. Once antigens are identified, antibodies can be produced through known methods in the art. The antibodies can be used for diagnostic purposes, e.g. identification of pathogens, or for immunological purposes, e.g. rational vaccine design. See for example J Virol Methods. 2009 December; 162(1-2):194-202. doi: 10.1016/j.jviromet.2009.08.006, which discloses antibody production from an antigen for the purposes of diagnostic applications against the influenza virus H5. Furthermore, in another preferred embodiment, the antibodies can be produced in applications of pathology in identifying pathogens. See Methods Mol Biol. 2009; 508:63-74. doi: 10.1007/978-1-59745-062-1_{—}6, which discloses the use of monoclonal antibodies for the detection of diseases in plant pathology.

Furthermore, in another embodiment, the present invention can be utilized for immunological purposes, for example, rational vaccine design. Rationally designed vaccines are composed of antigens, delivery systems, and often adjuvants that elicit predictable immune responses against specific epitopes to protect against a particular pathogen. See for example PLOS DOI: 10.1371/journal.ppat.100300, published Nov. 8, 2012, and PLOS DOI: 10.1371/journal.ppat.1002095, published Jun. 16, 2011, which discloses development of vaccines based upon rational design.

In another embodiment, as described in the preceding paragraphs, the mRNA of a pathogen isolated during active-blood borne infection can be captured, eluted and analyzed for use in the creation of a cDNA library. See BioTechniques 38:451-458, March, 2005, which details the construction of a cDNA library from RNA. See Genetics Nov. 1, 1992 vol. 132 no. 3 665-673, which details the use of a cDNA library for the identification of genes whose overexpression causes lethality in yeast. See also Büssow, Konrad, et al. Nucleic Acids Research 26.21 (1998): 5007-5008, which discloses a method for global protein expression and antibody screening utilizing a cDNA library. Knowing which proteins contribute to antibody responses allows one to generate tailored rational vaccines against a smaller subset of antigens. For example, restricting a vaccine to a protein antigen will favor a T-cell response over a humoral B-cell response.

It should be appreciated that with the above mentioned applications, data analysis can be accomplished by using any of a number of commercially available software packages available from Applied Math, Bio-Rad, BioSystematics, Media Cybernetics, or Scanalytics.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

### Sample

Blood samples from healthy volunteers were spiked with clinically relevant concentrations of bacteria (1-10 CFU/mL) including both laboratory strains and clinical isolates of the bacterial species most frequently found in bloodstream infections.

### Example 2

### Antibody Preparation

In order to generate polyclonal, pan-Gram-positive bacteria-specific IgG, a goat was immunized by first administering bacterial antigens suspended in complete Freund's adjuvant intra lymph node, followed by subcutaneous injection of bacterial antigens in incomplete Freund's adjuvant in 2 week intervals. The antigens were prepared for antibody production by growing bacteria to exponential phase (OD₆₀₀=0.4-0.8). Following harvest of the bacteria by centrifugation, the bacteria was inactivated using formalin fixation in 4% formaldehyde for 4 hr at 37° C. After 3 washes of bacteria with PBS (15 min wash, centrifugation for 20 min at 4000 rpm) the antigen concentration was measured using BCA assay and the antigen was used at 1 mg/mL for immunization. In order to generate Gram-positive bacteria-specific IgG, several bacterial species were used for inoculation: Staphylococcus aureus, Staphylococcus epidermidis, Enterococcus faecium and Enterococcus fecalis.

The immune serum was purified using affinity chromatography on a protein G sepharose column (GE Healthcare), and reactivity was determined using ELISA. Antibodies cross-reacting with Gram-negative bacteria and fungi were removed by absorption of purified IgG with formalin-fixed Gram-negative bacteria and fungi. The formalin-fixed organisms were prepared similar to as described above and mixed with IgG. After incubation for 2 hrs at room temperature, the preparation was centrifuged to remove bacteria. Final antibody preparation was clarified by centrifugation and used for the preparation of antigen-specific magnetic beads.

### Example 3

### Preparation of Antigen-Specific Magnetic Beads

Superparamagnetic beads were synthesized by encapsulating iron oxide nanoparticles (5-15 nm diameter) in a latex core and labeling with goat IgG. Ferrofluid containing nanoparticles in organic solvent was precipitated with ethanol, nanoparticles were resuspended in aqueous solution of styrene and surfactant Hitenol BC-10, and emulsified using sonication. The mixture was allowed to equilibrate overnight with stirring and filtered through 1.2 and 0.45 μm filters to achieve uniform micelle size. Styrene, acrylic acid and divynilbenzene were added in carbonate buffer at pH 9.6. The polymerization was initiated in a mixture at 70° C. with the addition of K₂S₂O₈ and the reaction was allowed to complete overnight. The synthesized particles were washed 3 times with 0.1% SDS using magnetic capture, filtered through 1.2, 0.8, and 0.45 μm filters and used for antibody conjugation.

The production of beads resulted in a distribution of sizes that may be characterized by an average size and a standard deviation. In the case of labeling and extracting of bacteria from blood, the average size for optimal performance was found to be between 100 and 350 nm, for example between 200 nm to 250 nm.

The purified IgG were conjugated to prepared beads using standard chemistry. After conjugation, the beads were resuspended in 0.1% BSA which is used to block non-specific binding sites on the bead and to increase the stability of bead preparation.

### Example 4

### Labeling of Rare Cells Using Excess of Magnetic Nanoparticles

Bacteria, present in blood during blood-stream infection, were magnetically labeled using the superparamagnetic beads prepared in Example 3 above. The spiked samples as described in Example 1 were diluted 3-fold with a Tris-based binding buffer and target-specific beads, followed by incubation on a shaking platform at 37° C. for up to 2 hr. After incubation, the labeled targets were magnetically separated followed by a wash step designed to remove blood products. See example 5 below.

### Example 5

### Magnetic Capture of Bound Bacteria

Blood including the magnetically labeled target bacteria and excess free beads were injected into a flow-through capture cell with a number of strong rare earth bar magnets placed perpendicular to the flow of the sample. With using a flow chamber with flow path cross-section 0.5 mm×20 mm (h×w) and 7 bar NdFeB magnets, a flow rate as high as 5 mL/min was achieved. After flowing the mixture through the channel in the presence of the magnet, a wash solution including heparin was flowed through the channel. The bound targets were washed with heparin-containing buffer one time to remove blood components and to reduce formation of magnetic particle aggregates. In order to effectively wash bound targets, the magnet was removed and captured magnetic material was resuspended in wash buffer, followed by re-application of the magnetic field and capture of the magnetic material in the same flow-through capture cell.

Removal of the captured labeled targets was possible after moving magnets away from the capture chamber and eluting with flow of buffer solution. The captured targets could then be used for gene expression analysis.

What is claimed is:

1. A method for analyzing a target, the method comprising
obtaining a sample comprising a target;
introducing a magnetic particle comprising a target-specific binding moiety to the sample, thereby forming a target/magnetic particle complex in the sample;
applying a magnetic field to facilitate isolation of the target from the sample; and
determining an expression profile of a nucleic acid derived from the target.

2. The method of claim 1, further comprising
flowing the sample through a channel comprising a second binding moiety attached to a surface of the channel; and
binding the target/magnetic particle complex to the surface of the channel, wherein the step of applying a magnetic field brings the target/magnetic particle complex into proximity of the surface to facilitate binding of the target/magnetic particle complex to the second binding moiety; and
washing away unbound magnetic particles and unbound sample components.

3. The method of claim 1, wherein the target comprises a pathogen.

4. The method of claim 3, wherein the pathogen comprises a bacterium, a virus, or a microorganism that can cause disease.

5. The method of claim 4, wherein the bacterium is a gram positive bacterium.

6. The method of claim 4, wherein the bacterium is a gram negative bacterium.

7. The method of claim 1, wherein the step of determining comprises conducting an assay selected from the group consisting of microarray analysis, sequencing, electrophoresis, RT-PCR, and a combination thereof.

8. The method of claim 1, wherein the target-specific binding moiety and the second binding moiety are selected from the group consisting of antibodies, receptors, aptamers, proteins, and ligands.

9. The method of claim 1, wherein the sample is selected from the group consisting of blood, sputum, urine, saliva, and sweat.

10. A method for analyzing a pathogen, the method comprising
obtaining a sample comprising a pathogen;
exposing the sample to a cocktail comprising a plurality of sets of magnetic particles to form pathogen/magnetic particle complexes in the sample, magnetic particles of each set being conjugated to a binding moiety specific to a pathogen; and
applying a magnetic field to capture pathogen/magnetic particles complexes on a surface;
washing away unbound particles and unbound components of the sample with a wash solution that reduces particle aggregation, thereby isolating the pathogen/magnetic particle complexes; and
determining an expression profile of a nucleic acid derived from the pathogen.

11. The method of claim 10, wherein the surface comprises a plurality of binding moieties, and the step of applying magnetic fields comprises applying alternating magnetic fields to bring the pathogen/magnetic particle complexes into proximity of the surface to facilitate binding of the pathogen/magnetic particle complexes to the plurality of binding moieties.

12. The method of claim 10, wherein the plurality of magnetic particles is differently functionalized so that the plurality of magnetic particles binds to a plurality of different pathogens.

13. The method of claim 10, wherein the pathogen comprises a bacterium, a virus, or any other microorganism that can cause disease.

14. The method of claim 13, wherein the bacterium is a gram positive bacterium.

15. The method of claim 13, wherein the bacterium is a gram negative bacterium.

16. The method of claim 10, wherein the step of determining comprises conducting an assay selected from the group consisting of microarray analysis, sequencing, RT-PCR, and a combination thereof.

17. The method of claim 1, wherein the binding moiety specific to the pathogen and the plurality of binding moieties are selected from the group consisting of antibodies, receptors, aptamers, proteins, and ligands.

18. The method of claim 1, wherein the sample is selected from the group consisting of blood, sputum, urine, saliva, and sweat.
